(19) United States
(12) Patent Application Publication
(10) Pub. No.: US 2017/0315047 A1
(43) Pub. Date: Nov. 2, 2017

(54) PRETREATMENT APPARATUS AND SAMPLE ANALYZER

(71) Applicant: Sysmex Corporation, Kobe-shi (JP)

(72) Inventor: Kazuhiro YAMADA, Kobe-shi (JP)

(21) Appl. No.: 15/497,584

(22) Filed: Apr. 26, 2017

(30) Foreign Application Priority Data
Apr. 28, 2016 (JP) 2016-091889

(51) Int. Cl. G01N 15/14; G01N 1/38; G01N 1/40; G01N 33/53; G01N 35/10; G01N 35/02; G01N 33/569; B04B 5/04; G01N 15/10; G01N 15/00

(52) U.S. Cl. CPC G01N 15/1436; G01N 35/1002; G01N 1/38; G01N 1/4077; G01N 33/5304; G01N 35/1095; B04B 5/0414; G01N 35/025; G01N 33/56972; G01N 2001/4083; G01N 2015/0065; G01N 2015/008; G01N 2015/1006

(57) ABSTRACT

A pretreatment apparatus includes a sample dispensing part 240, a reagent dispensing part 280 for dispensing a labeling reagent, a first process part 210 having a centrifuge device for performing a centrifugation process, a second process part 220, and a control part for distributing the dispensing destination of the sample to either the first sample container 217 or the second sample container 227 according to whether centrifugation process is required for the sample.

### CROSS REFERENCE TO RELATED APPLICATIONS

This application claims priority from prior Japanese Patent Application No. 2016-091889, filed on Apr. 28, 2016, entitled, PRETREATMENT APPARATUS AND SAMPLE ANALYZER”, the entire contents of which are incorporated herein by reference.

### FIELD OF THE INVENTION

The present invention relates to a pretreatment apparatus for preparing a measurement sample from a specimen, and a sample analyzer provided with the pretreatment apparatus.

### BACKGROUND

In flow cytometry in which a measurement object is optically analyzed, pretreatment is required to prepare the measurement sample. A process is performed in this pretreatment to mix the sample containing the substance to be measured with a labeling reagent that labels the substance to be measured. The substance to be measured is labeled by the labeling reagent through the mixing of the sample and the labeling reagent.

In the pretreatment for flow cytometry, centrifugation process of a mixed solution of a sample and a labeling reagent may be required. U.S. Pat. No. 9,557,249 discloses a system that includes a centrifugation section and performs pretreatment automatically. In the system of U.S. Pat. No. 9,557,249, pretreatment including centrifugation is performed in the centrifugation section. That is, centrifugation is performed by the centrifugation section after whole blood has been dispensed to a sample container installed in the centrifugation section, and reagent has been dispensed to the whole blood.

### SUMMARY OF THE INVENTION

In the case of using the system of U.S. Pat. No. 9,557,249, it is possible to perform pretreatment of a sample that does not require centrifugation in the centrifugation section insofar as the centrifugation section of the system is not caused to function. However, since it is not possible to simultaneously process a sample requiring centrifugation and a sample not requiring centrifugation in the centrifugation section in the system, the user must select only samples that do not require centrifugation or only samples that require centrifugation and supply them to the system.

A first aspect of the invention is a pretreatment apparatus comprising: a sample dispensing part configured to dispense a sample containing a measurement object; a reagent dispensing part configured to dispense labeling reagent to label the measurement object; a first process part including: a holder configured to hold one or more first sample containers to which the sample and the labeling reagent are to be dispensed; and a centrifuge device configured to perform a centrifuge process on a mixed solution in the first sample container held by the holder; a second process part including: a holder configured to hold one or more second sample containers to which the sample and the labeling reagent are to be dispensed; and a control part programmed to: control the sample dispensing part so as to dispense the sample to either the first sample container or the second sample container according to need of centrifugation process on the sample; control the reagent dispensing part so as to dispense the labeling reagent to a sample container into which the sample is dispensed; control the first process part to prepare a first measurement sample for flow cytometric analysis by preparing a first mixed solution in the first sample container from the sample and the labeling reagent dispensed in the first sample container, and by performing the centrifugation process on the first mixed solution; and control the second process part to prepare a second measurement sample for flow cytometric analysis by preparing a second mixed solution in the second sample container from the sample and the labeling reagent dispensed in the second sample container.

A second aspect of the invention is a pretreatment apparatus comprising: a sample dispensing part configured to dispense a sample containing a measurement object; a reagent dispensing part configured to dispense labeling reagent to label the measurement object; a first process part including: a holder configured to hold one or more first sample containers; and a centrifuge device configured to perform a centrifugation process on a mixed solution in the first sample container held by the holder; a second process part including a holder for holding one or more second sample containers; and a control part programmed to: control the sample dispensing part so as to dispense the sample to the second sample container; control the reagent dispensing part so as to dispense the labeling reagent to the second sample container; control the second processing unit so as to prepare a mixed solution in the second sample container from the sample and the labeling reagent dispensed in the second sample container; and perform controls to transfer the mixed solution from the second sample container to the first sample container when a centrifugation process on the mixed solution in the second sample container is required, and perform centrifugation process on the mixed liquid by the first processing part. In an embodiment, the first processing section includes a holder for holding one or more first sample containers to which a sample and a labeling reagent are dispensed, and a centrifugation device for performing a centrifugation process on a mixed solution in a first sample container held in the holder.

### BRIEF DESCRIPTION OF THE DRAWINGS

FIG. 1 is a plane view (A-A arrow view of FIG. 2A) of a pretreatment apparatus;

FIG. 2A is a side view of a sample analyzer;

FIG. 2B is a plane view (B-B arrow view of FIG. 2A) of the sample analyzer;

FIG. 3 is a brief structural view of the pretreatment apparatus and the measuring part;

FIG. 4 illustrates the pretreatment process;

FIG. 5 is a structural view of the controller;

FIG. 6 is a timing chart of the pretreatment process;

FIG. 7 is a flow chart of the stirring process;

FIG. 8 is a data structure diagram of the sample information;

FIG. 9 is a flow chart of the sample dispensing process;

FIG. 10 is a flow chart of the labeling reagent dispensing process;

FIG. 11 is a flow chart of the hemolytic agent dispensing process;

FIG. 12 is a flow chart of the centrifugation process;

FIG. 13 is a flow chart of the supernatant removal/buffering solution dispensing process;

FIG. 14 is a flow chart of the transport process/measuring process;

FIG. 15 is a flow chart of the sample dispensing process;

FIG. 16 is a flow chart of the labeling reagent dispensing process;

FIG. 17 is a flow chart of the hemolytic agent dispensing process; and

FIG. 18 is a flow chart of the hemolytic reaction termination process.

### DESCRIPTION OF THE EMBODIMENTS OF THE INVENTION

### 1. Sample Analyzer

A sample analyzer 100 analyzes samples that contain a measurement object. The sample analyzer 100 analyzes a sample by flow cytometry. The measurement object, for example, may be a cell, and more specifically, for example, a cell surface antigen. A sample containing cells, for example, may be a blood sample, and more specifically, for example, a whole blood sample.

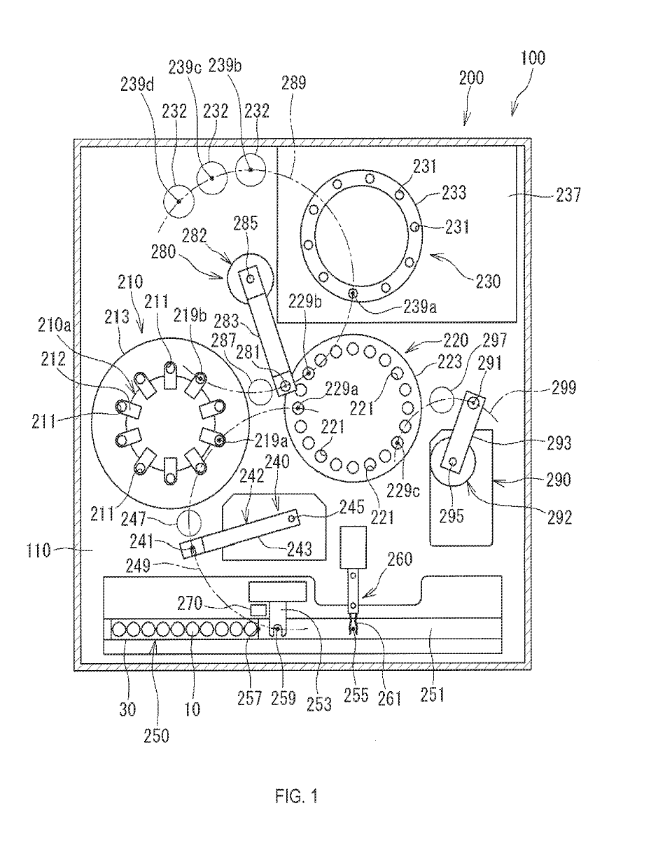

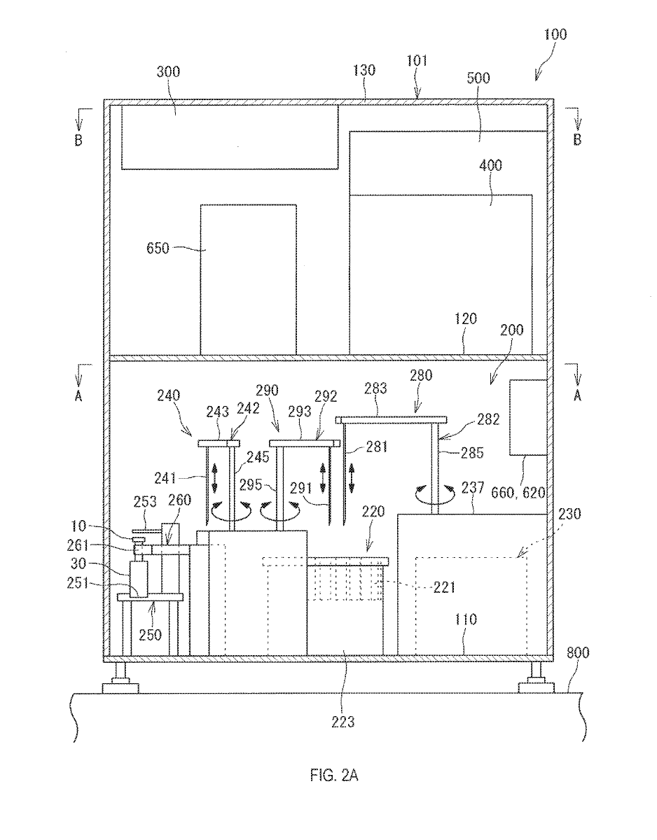

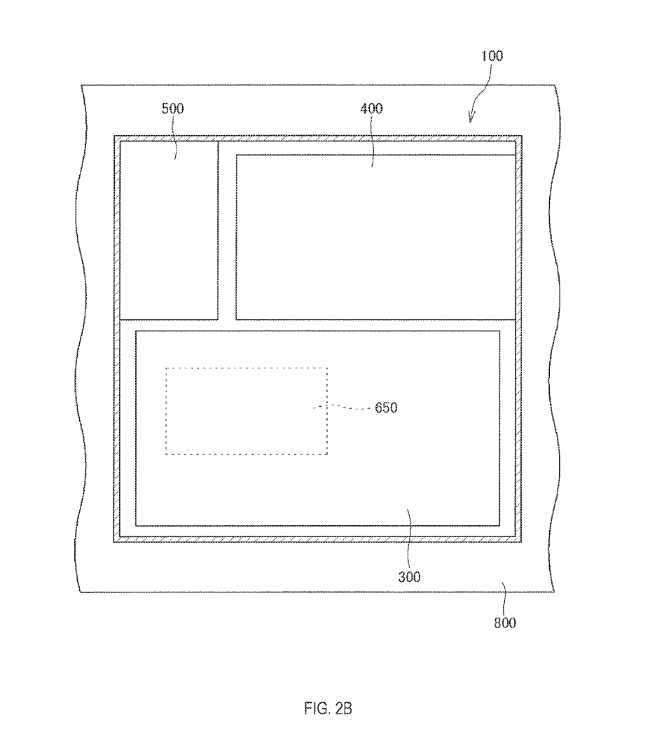

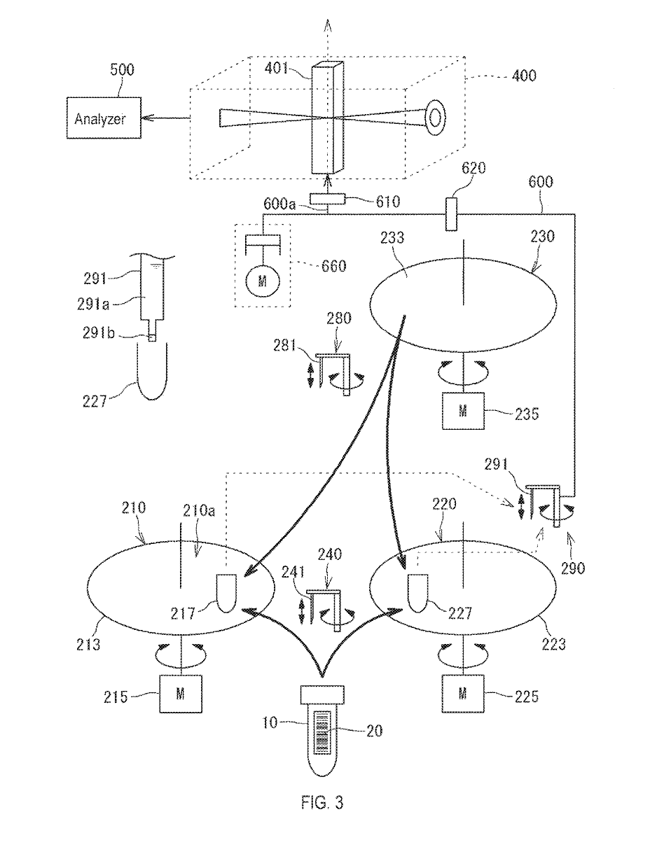

The sample analyzer 100 includes the pretreatment apparatus 200 shown in FIG. 1. The pretreatment apparatus 200 performs pretreatment processing to prepare a measurement sample for flow cytometric analysis from a sample. As shown in FIG. 2A, FIG. 2B, and FIG. 3, the sample analyzer 100 also includes a measuring part 400 and analyzing part 500. The measuring part 400 optically measures the sample that was prepared by the pretreatment apparatus 200. The analyzing part 500 analyzes the measurement results output from the measuring part 400.

The pretreatment apparatus 200 shown in FIG. 1 includes a plurality of processing parts 210 and 220. The processing parts 210 and 220 respectively perform pretreatment. The pretreatment is a process for preparing a measurement sample from a sample. The plurality of processing parts include a centrifuge-reaction process part 210 and a reaction process part 220. The centrifuge-reaction process part 210 performs a pretreatment that includes a centrifugation process. The reaction process part 220 performs a pretreatment that does not include a centrifugation process. Pretreatment invariably includes a sample and reagent reaction process.

The centrifuge-reaction process part 210 is provided with a circular turntable 213. The centrifuge-reaction process part 210 has a centrifuge device 210a that performs the centrifugation process, and the centrifuge device 210a rotates the turntable 213. The turntable 213 has holders 211. The holder 211 holds a sample container 217 to which the sample and reagent are dispensed. A first sample is prepared within the sample container 217. The holder 211 has an insertion hole, and the inserted sample container 217 is held in the insertion hole. A plurality of holders 211 are provided in the turntable 213, and one or more sample containers 217 may be held in the turntable 213. The plurality of holders 221 are arranged circumferentially. For example, ten holders 211 may be provided.

The centrifuge device 210a has a motor 215. As shown in FIG. 3, the rotating table 213 is rotated by driving the motor 215 to turn. The motor 215 rotates the rotating table 213 to position the sample container 217, and rotates the turntable 213 for centrifugation processing on the liquid in the sample container 217. The centrifugation device 210a is provided with a lid 212 for closing the upper end opening of the sample container 217 held by the holder 211. The lid 212 prevents the sample from leaving the sample container 217 when the turntable 213 is rotating for centrifugation.

The centrifuge-reaction process part 210 is provided with a circular turntable 223. The turntable 223 has holders 221. The holder 221 holds a sample container 227 to which the sample and reagent are dispensed. A second sample is prepared within the sample container 227. The holder 221 has an insertion hole, and the inserted sample container 227 is held in the insertion hole. A plurality of holders 221 are provided in the turntable 223, and one or more sample containers 227 may be held in the turntable 223. The plurality of holders 221 are arranged circumferentially. More holders 221 may be provided than the holders 211, for example, twenty may be provided.

As shown in FIG. 3, the turntable 223 is rotated by driving the motor 225 to turn. The motor 225 rotates the turntable 223 to position the sample container 227.

The pretreatment apparatus 200 shown in FIG. 1 includes a reagent holding part 230. The reagent holding part 230 is provided with a circular turntable 233. The turntable 233 has reagent holders 231. The reagent holder 231 holds a reagent container that contains reagent. In the embodiment, the reagent in the reagent container held in the reagent holder 231 is labeling reagent.

The labeling reagent, for example, includes labeled antibody reagent. The labeled antibody reagent is a reagent that labels the antigen to be measured by antigen-antibody reaction. The labeled antibody reagent has a monoclonal antibody. A monoclonal antibody with a CD number corresponding to the surface antigen to be measured is used. In embodiment, a plurality of labeled antibody reagents are used for measurement of various surface antigens. A plurality of labeling reagents can be held since the turntable 233 includes a plurality of reagent holders 231. The plurality of holders 231 are arranged circumferentially. For example, ten holders 231 may be provided.

As shown in FIG. 3, the turntable 233 is rotated by driving the motor 235. The motor 235 rotates the turntable 233 to position the reagent container. The turntable 233 is provided within a cold storage part 237. The cold storage part 237 refrigerates the reagent.

The reagent holding part 230 maintains the reagent holders 232 arranged outside the cold storage part 237. The reagent holder 232 holds a reagent container that contains reagent. A plurality of reagent holders 232 are provided. In the embodiment, the reagent in the reagent container held in the reagent holder 232 is hemolytic agent. The reagent in the other reagent container held in the reagent holder 232 is a buffer solution. Note that hemolytic agent and buffer agent are preferably disposed outside the cold insulation part 237 since these reagents do not require cold storage. The plurality of reagent holders 232 are disposed along the arcing track 289 of the reagent nozzle 281.

The pretreatment apparatus 200 includes a sample dispensing part 240 that dispenses sample to the centrifuge-reaction process part 210 or reaction process part 220. The sample dispensing part 240 has a sample nozzle 241. The sample nozzle 241 suctions and discharges the sample. The sample nozzle 241 moves along the arcing track 249 shown in FIG. 1 by a moving device 242. The moving device 242 includes an arm 243 that holds the sample nozzle 241, and a drive shaft 245 that drives the arm 243. The drive shaft 245 is rotationally driven around the vertical center axis. The sample nozzle 241 moves along the arcing track 249 due to the rotation of the drive shaft 245. The drive shaft 245 is also driven so as to move in a vertical direction. Due to the vertical movement of the drive shaft 245, the sample nozzle 241 moves in the vertical direction. The sample nozzle 241 moves forward and backward into the sample container 10 or the reagent containers 217 and 227 by the vertical movement.

The sample nozzle 241 is cleaned by a sample nozzle cleaning tank 247. The cleaning tank 247 is arranged at a position in the arcing track 249. The cleaning tank 247 has an opening at the top into which the sample nozzle 241 is inserted. The sample nozzle 241 is cleaned by a cleaning liquid in the cleaning tank 247. The cleaning liquid may be, for example, a sheath fluid used to flow a sample to a flow cell 401, which will be described later. The cleaning liquid is sprayed onto the outer wall of the sample nozzle 241 inserted into the cleaning tank 247 to clean the outer wall of the sample nozzle 241. The sample nozzle 241 further discharges cleaning liquid to the cleaning tank 247, whereby the inside of the sample nozzle 241 is also cleaned. Cleaning of the sample nozzle 241 is performed every time the sample nozzle 241 suctions and discharges a sample, to prevent contamination between samples.

The pretreatment apparatus 200 is provided with a transport part 250 for transporting the sample container 10 containing the sample. The transport part 250 transports the sample rack 30 holding a plurality of sample containers 10. The transport part 250 includes a linear transport path 251. The sample rack 30 placed on the transport path 251 moves along the transport path 251 by a transport device (not shown).

In the embodiment, a stirring standby position 255, a barcode reading position 257, and a sample suctioning position 259 are set on the transport path 251. The stirring standby position 255 is a position where the sample waits until stirred by the stirring part 260. The stirring part 260 grips the sample container 10 at the stirring standby position 255 by the holding part 261 and stirs the sample in the sample container 10. The barcode reading position 257 is a position at which the barcode 20 affixed to the sample container 10 is read by the barcode reader 270. The sample suctioning position 259 is a position where the sample is suctioned by the sample nozzle 241. The sample nozzle 241 suctions the sample in the sample container 10 at the sample suctioning position 259. The sample suctioning position 259 is at a position where it intersects the arcing track 249 of the sample nozzle 241 and the transport path 251.

Note that a lid pressing part 253 is disposed above the sample container 10 at the sample suction position 259. The lid pressing part 253 prevents the lid that closes the upper opening of the sample container 10 from being detached due to ascent of the sample nozzle 241 penetrating the lid.

In addition to the sample suction position 259, a sample dispensing position 219a on the centrifuge-reaction process part 210 and a sample dispensing position 229a on the reaction process part 220 are set in the arcing track 249 of the sample nozzle. The sample dispensing position 219a is a position where the sample nozzle 241 dispenses the sample into the centrifuge-reaction process part 210. The sample container 217 into which the sample is to be dispensed is moved to the sample dispensing position 219a by the rotation of the turntable 213. The sample dispensing position 229a is a position where the sample nozzle 241 dispenses the sample into the reaction process part 220. The sample container 227 into which the sample is to be dispensed is moved to the sample dispensing position 229a by the rotation of the turntable 223.

The pretreatment apparatus 200 includes a reagent dispensing part 280 that dispenses reagent to either the centrifuge-reaction process part 210 or reaction process part 220. The reagent dispensing part 280 includes a reagent nozzle 281. The reagent nozzle 281 suctions and discharges the reagent. The reagent nozzle 281 moves along the arcing track 289 shown in FIG. 1 by a moving device 282. The moving device 282 includes an arm 283 that holds the reagent nozzle 281, and a drive shaft 285 that drives the arm 283. The drive shaft 285 is rotationally driven around the vertical center axis. The reagent nozzle 281 moves along the arcing track 289 due to the rotation of the drive shaft 285. The drive shaft 285 is also driven so as to move in a vertical direction. Due to the vertical movement of the drive shaft 285, the reagent nozzle 281 moves in the vertical direction. The reagent nozzle 281 moves forward and backward into the sample containers 217 and 227 or the reagent container by the vertical movement of the reagent nozzle.

The reagent nozzle 281 is cleaned by a sample nozzle cleaning tank 287. The cleaning tank 287 is arranged at a position in the arcing track 289. The cleaning tank 287 has an opening at the top into which the reagent nozzle 281 is inserted. The reagent nozzle 281 is cleaned by a cleaning liquid in the cleaning tank 287. The nozzle cleaning method used in the cleaning tank 287 is the same as the cleaning method of the sample nozzle 241. Cleaning of the reagent nozzle 281 is performed every time the reagent nozzle 281 suctions and discharges a reagent or a sample to prevent contamination between reagents or samples.

In the embodiment, the reagent suction position 239a, the reagent suction positions 239b, 239c, and 239d, the reagent dispensing position 219b, and the reagent dispensing position 229b are set in the arcing track 289 of the reagent nozzle. The reagent suction position 239a is a position at which the reagent nozzle 281 suctions the labeling reagent. The first reagent container containing the suctioned labeling reagent moves to the reagent suction position 239a by the rotation of the turntable 233. The reagent suction positions 239b, 239c, and 239d are positions where the reagent nozzle 281 suctions the hemolytic agent or the buffer solution. The reagent container containing the hemolytic agent to be suctioned or the reagent container containing the buffer solution moves to the suction positions 239b, 239c, 239d by the rotation of the rotary table 233.

The reagent dispensing position 219b is a position where the reagent nozzle 281 dispenses the reagent in the centrifuge-reaction process part 210. The sample container 217 into which the sample is to be dispensed is moved to the sample dispensing position 219a by the rotation of the turntable 213. The reagent dispensing position 229b is a position where the reagent nozzle 281 dispenses the reagent in the centrifuge-reaction process part 210. The sample container 227 into which the reagent is to be dispensed is moved to the reagent dispensing position 229a by the rotation of the turntable 223.

Note that the reagent dispensing part 280 also plays a role of transferring the sample prepared in the centrifuge-reaction process part 210 from the centrifuge-reaction process part 210 to the reaction process part 220 in order to transfer it to the measurement unit 400. The transfer of the sample is described later.

The pretreatment device 200 includes a sample suction part 290 that suctions the measurement sample in the reaction process part 220. The sample suction part 290 has a sample nozzle 291. The sample nozzle 291 suctions the measurement sample. The sample nozzle 291 moves along the arcing track 299 shown in FIG. 1 by a moving device 292. The moving device 292 includes an arm 293 that holds the reagent nozzle 291, and a drive shaft 295 that drives the arm 293. The drive shaft 295 is rotationally driven around the vertical center axis. The sample nozzle 291 moves along the arcing track 299 due to the rotation of the drive shaft 295. The drive shaft 295 is also driven so as to move in a vertical direction. Due to the vertical movement of the drive shaft 295, the sample nozzle 291 moves in the vertical direction. The sample nozzle 291 moves forward and backward into the sample container 227 by the vertical movement of the sample nozzle.

The sample nozzle 291 is cleaned by the sample nozzle cleaning tank 297. The cleaning tank 297 is arranged at a position in the arcing track 299. The cleaning tank 297 has an opening at the top into which the sample nozzle 291 is inserted. The sample nozzle 291 is cleaned by a cleaning liquid in the cleaning tank 297. The nozzle cleaning method used in the cleaning tank 297 is the same as the cleaning method of the sample nozzle 241. Cleaning of the sample nozzle 291 is performed every time the sample nozzle 291 suctions and discharges a reagent to prevent contamination between samples.

In the embodiment, the sample suction position 229c is set in the arcing track 299 of the sample nozzle 291. The sample suction position 229c is a position at which the sample nozzle 291 suctions the measurement sample. The sample container 227 containing the measurement sample to be suctioned by the sample nozzle 291 moves to the sample suction position 229c by the rotation of the turntable 223. Here, the measurement sample to be suctioned by the sample nozzle 291 is either a sample prepared in the reaction process part 220, or a sample prepared in the centrifuge-reaction process part 210 and transferred to the sample container 227 of the reaction process part 220.

As shown in FIG. 2A, the sample analyzer 100 includes a frame 101 that has a multi-stage structure with a lower stage and an upper stage. Inside the frame 101, a pretreatment apparatus 200, a measuring part 400, an analyzing part 500 and the like are arranged inside the frame 101. The frame 101 includes a base 110 for supporting each member constituting the pretreatment apparatus 200, and a base 120 for supporting the measuring part 400 and the analyzing part 500 and the like. The space between the base 110 and the base 120 is a layout space for each member constituting the pretreatment apparatus 200. The space between the base part 120 and the upper part 130 of the frame 101 is the layout space of the measuring part 400 and the analyzing part 500.

The measuring part 400 optically measures the sample. The measuring part 400 has a flow cell 401. The sample supplied to the flow cell 401 is irradiated with light from a light source, and a detection part receives the light emitted from the sample. The detection part detects the fluorescent light given off from the sample. The detection part also can detect the scattered light from the sample. The measuring part 400 outputs the received light signals as measurement signals to the analyzing part 500. The analyzing part 500 analyzes the measurement signals, and outputs the analysis result of the sample. The analyzing part 500 is configured by a computer.

A control part 300 and a fluid circuit part 650 are also arranged in the layout space between the base part 120 and the upper part 130 of the frame 101. The control part 300 controls the pretreatment apparatus 200 and the measuring part 400. The fluid circuit part 650 includes a solenoid valve 610 for controlling the flow of the fluid, a pump and the like, and performs operations for transferring cleaning liquid, samples and the like in the pretreatment apparatus 200. The sample suction part 290 and the measuring part 400 are connected by a flow path that becomes the sample transfer path 600. Solenoid valves 610 and 620 are provided in the middle of the sample transfer path 600. One end of the flow path 600a is connected to the sample transfer path 600 at a position between the electromagnetic valves 610 and 620. A pump 660 is connected to the other end of the flow path 600a. The pump 660 suctions a certain amount of sample from the sample nozzle 291 and supplies the suctioned sample to the flow cell 401. The pump is, for example, a syringe pump. In the present embodiment, the sample suction part 290, the flow paths 600 and 600a, and the pump 660 configure a transfer part for transferring the measurement sample to the measuring part 400.

In this embodiment, two process parts 210 and 220 are provided, but the sample analyzer 100 overall is rendered compact. For example, the sample dispensing part 240 can access the two process units 210 and 220 and the sample container 10 by a simple operation along the arcing track 249 of the sample nozzle 241. The reagent dispensing part 280 also can access the two process units 210 and 220 and the reagent container by a simple operation along the arcing track 289 of the reagent nozzle 281. Therefore, the moving devices 242 and 282 of the nozzles 241 and 281 may be simple devices. In addition, since the reagent dispensing part 280 is responsible for transferring the sample or the mixture during preparation between the centrifuge-reaction process part 210 and the reaction process part 220, a mechanism for this transfer is unnecessary.

Furthermore, in the present embodiment, the size in the plan view can be rendered compact and the sample analyzer 100 can be installed in a small installation space since the measuring part 400 and the like are arranged above the pretreatment apparatus 200. As shown in FIGS. 2A and 2B, the sample analyzer 100 is installed, for example, on the mounting surface 800 of a shelf or a cabinet. Since the sample analyzer 100 is compact, the space occupied by the mounting surface 800 can be advantageously reduced. The measuring part 400 also may be arranged below the pretreatment apparatus 200. When the measuring part 400 is disposed above or below the pretreatment apparatus 200, for example, it is possible to suppress interruption of user's work by each part of the pretreatment apparatus 200 when adjusting the optical axis of the measuring part 400, hence the work is easy. Further, when the pretreatment apparatus 200 is disposed below the measuring part 400, the transport part 250 is positioned near waist height of the user, and the user can easily install the rack 30 in the transport part 250 since the sample analyzer 100 is mounted on the cabinet.

As described above, most of the fluid circuit elements for the pretreatment apparatus 100 are provided in the fluid circuit part 650 arranged in the upper layout space. As shown in FIG. 2A, the pump 660 and solenoid valve 620 are arranged in the layout space of the lower stage the same as the sample nozzle 291. In addition, the flow paths 600 and 600a connecting the sample nozzle 291 and the pump 660 are also arranged in the lower layout space As a result, the distance from the pump 660 for quantifying the sample to the sample nozzle 291 is reduced, which makes it possible to advantageously quantify quickly or accurately. That is, as shown in FIG. 3, when the sample nozzle 291 suctions the sample, the interior of the sample nozzle 291 is filled with the sheath liquid 291a in advance, and an air gap 291b is formed at the tip of the sample nozzle 291. Then, by moving the sheath liquid 291a and the air gap 291b in the sample nozzle 291 by the suction action of the pump 660, the sample is suctioned from the tip of the sample nozzle 291. At this time, if the distance from the pump 660 to the sample nozzle 291 is large, the volume of the sheath liquid in between is increase, and the sheath liquid does not move with good reaction even if the pump 660 is operated, and the quantitative accuracy also decreases. However, if the distance from the pump 660 to the sample nozzle 291 is small, the quickness and accuracy of quantification can be enhanced.

### 2. Pretreatment

### 2.1 Summary of Pretreatment

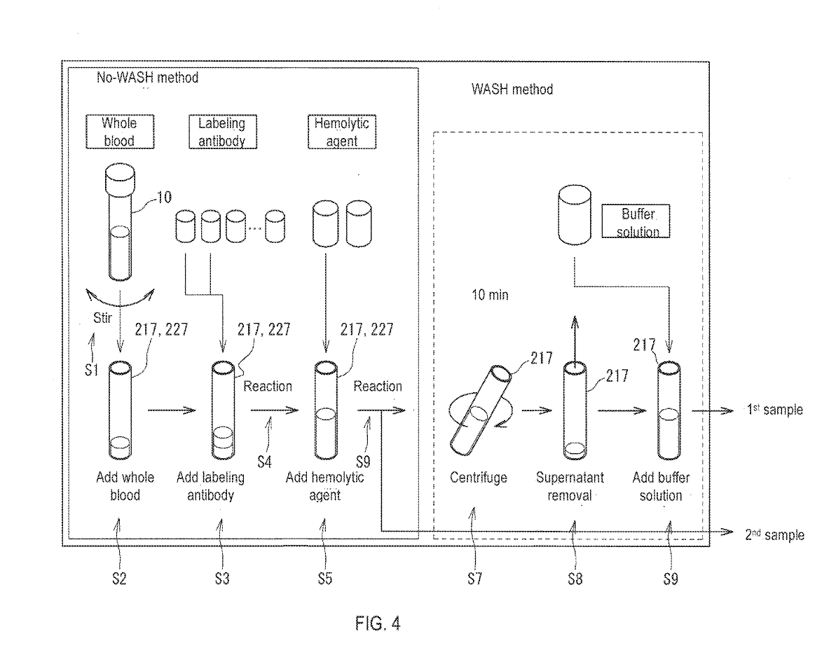

FIG. 4 shows two types of pretreatment. The two types of pretreatment are distinguished by the presence or absence of centrifugation process. The pretreatment including the centrifugation process is called the WASH method and corresponds to the pretreatment performed in the centrifuge-reaction process part 210 described above. The pretreatment not including the centrifugation process is called the No-WASH method, and corresponds to the pretreatment performed in the above-described reaction process part 220.

The No-WASH method is performed, for example, as a pretreatment for lymphocyte subset analysis. The measurement subjects in the lymphocyte subset analysis are, for example, T cell (CD3), B cell (CD 19 or CD 20), helper T cell (CD4), suppressor/cytotoxic T cell (CD8), natural killer (NK) cell (CD 56) and the like. In the measurement sample preparation for the analysis of the lymphocyte subset, it is sufficient that treatment with a hemolytic agent suffices to remove unnecessary substances, so that centrifugation may be omitted.

The WASH method is performed, for example, as pretreatment for leukemia typing analysis. In preparing a measurement sample for leukemia typing analysis, removal of unnecessary materials with a hemolytic agent is insufficient for treatment, so that centrifugation process is performed.

The No-WASH method and the WASH method each include a plurality of steps. In FIG. 4, the WASH method includes the steps from step S1 to step S9. In FIG. 4, the No-WASH method includes the steps from step S1 to step S6, but does not include steps S7 and later that constitute a centrifugation process.

In step S1, a stirring process is performed. In the stirring process, the sample held in the sample container 10 is stirred. In step S2, the sample dispensing process is performed. In the sample dispensing process, the mixed sample is dispensed from the sample container 10 to the sample containers 217 and 227.

In step S3, the labeling reagent dispensing process is performed. In the labeling reagent dispensing process, the labeling reagent is dispensed to the samples containers 217 and 227 containing the sample. In step S4, the reaction treatment is performed. The reaction treatment here is a treatment for reacting the specimen with the labeling reagent. In the reaction treatment of the labeling reagent, it is only necessary to wait for the time required for the reaction, such as an antigen-antibody reaction, to elapse. Heating also may be performed in the labeling reagent reaction treatment.

In step S5, the hemolytic agent dispensing process is performed. In the hemolytic agent dispensing process, the hemolytic agent is dispensed to the sample containers 217 and 227 in which the reaction treatment of step S4 has been completed. In step S6, the reaction treatment is performed. The reaction treatment here is a hemolysis treatment with a hemolytic agent. In the reaction treatment of the hemolytic agent, it is only necessary to wait for the time required for hemolysis. Heating also may be performed in the hemolytic agent reaction treatment.

The No-WASH method ends when the reaction treatment of step S6 is completed. That is, the preparation of the sample is completed when step S6 is completed. That is, the preparation of the sample is completed when step S6 is completed. No centrifugation process is performed on the mixture. On the other hand, in the WASH method, after the reaction treatment in step S6, steps S7 to S9 continue.

In the WASH method, in step S7, a centrifugation process is performed on the mixed solution in which the sample and the reagent are mixed. In step S8, supernatant removal treatment is performed. The supernatant contains substances that become unnecessary for measurement, such as hemolyzed erythrocyte debris, the remaining labeling reagent that has not bound to the surface antigen. In the supernatant removal treatment, the supernatant liquid generated by the centrifugation process is removed from the sample container 217. In step S9, the buffer solution dispensing process is performed. In the buffer solution dispensing process, buffer solution is dispensed to the sample container 217 from which the supernatant liquid has been removed. The WASH method ends when the buffer solution dispensing process of step S9 is completed.

### 2.2 Control of the Pretreatment Apparatus

The pretreatment apparatus 200 can perform a plurality of types of pretreatment including pretreatment by the WASH method and pretreatment by the No-WASH method. Pretreatment is controlled by the control part 300. The control part 300 of the embodiment controls the operations of the sample dispensing part 240, the reagent dispensing part 280, and the sample suction part 290. The control part 300 of the embodiment also controls the motor 215 of the centrifuge-reaction process part 210, the motor 225 of the reaction process part 220, and the motor 235 of the reagent holding part 230.

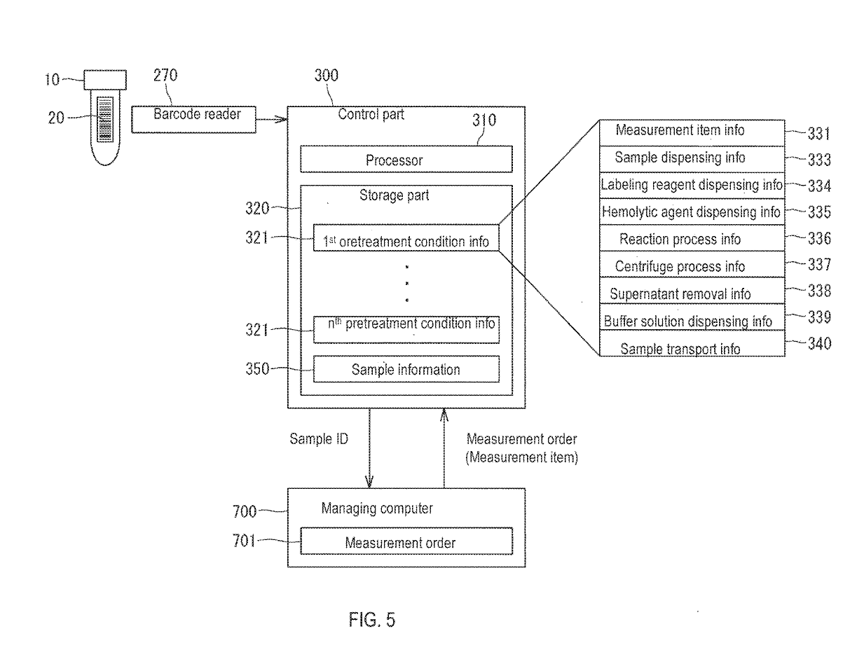

The control part 300 is configured by a computer. As shown in FIG. 5, the control part 300 has a processor 310 and a memory part 320. A computer program for controlling the pretreatment is stored in the memory part 320. The processor 310 executes the computer program.

The storage part 320 can store one or more pretreatment condition information 321. In the storage part 320 shown in FIG. 5, n pieces of pretreatment condition information 321 (n is an integer of 1 or more) are stored. The pretreatment condition information 321 indicates the protocol at the time of performing pretreatment. The processor 310 controls pretreatment according to pretreatment condition information 321. The pretreatment condition information 321 may be preset in the storage part 320 or may be set in the storage part 320 by user input.

As pretreatment, whether the WASH method or the No-WASH method should be performed will differ depending on the type of measurement item that is to be measured and analyzed. The measurement items include, for example, a T cell subset, a lymphocyte subset, a white blood cell primary panel, an AML secondary panel, a B-ALL secondary panel, a T-ALL secondary panel. The type of reagent used for pretreatment, the presence or absence of centrifugation, etc. will differ depending on the measurement item. In order to enable different pretreatments according to the measurement item, the pretreatment condition corresponding to the measurement item is set in the pretreatment condition information 321.

Pretreatment condition information 321 includes the information 331 to 340 shown in FIG. 5. For example, pretreatment condition information 321 includes measurement item information 331. The measurement item information 331 is, for example, information indicating the name or identifier of the measurement item.

The sample dispensing process information 333 in the pretreatment condition information 321 includes, for example, information indicating the amount of the sample to be dispensed from the sample container 10 to the sample containers 217 and 227. The labeling reagent dispensing process information 334 includes, for example, information indicating the name or identifier of the labeling reagent and information indicating the amount of the labeling reagent to be dispensed. Since a plurality of labeling reagents may be used for one measurement item, the labeled reagent dispensing process information 334 may include information on a plurality of labeling reagents.

The hemolytic agent dispensing process information 335 includes, for example, information indicating the name or identifier of the hemolytic agent and information indicating the amount of the hemolytic agent to be dispensed. The reaction process information 336 includes, for example, information indicating the reaction time (for example, antigen-antibody reaction time) of the labeling reagent and information indicating the reaction time (for example, hemolysis process time) of the hemolytic agent.

The centrifuge process information 337 includes, for example, information indicating whether to perform a centrifuge process, information indicating a time of a centrifugation process, and information indicating a rotation speed for centrifugation.

The supernatant removal process information 338 includes, for example, information indicating whether to perform the supernatant removal process, and setting information of a supernatant removal method. The buffer solution dispensing process information 339 includes, for example, information indicating whether to perform a buffer solution dispensing process, information indicating a name or an identifier of a buffer solution, and information indicating a dispensing amount of a buffer solution. The sample transport process information 340 includes, for example, information indicating whether to transport the sample from the pretreatment apparatus 200 to the measuring part 400, and information indicating a transport speed.

In the present embodiment, the pretreatment of the WASH method is performed according to the pretreatment condition information 321 including the centrifuge process information 337 set to perform the centrifuge process. On the other hand, the pretreatment of the No-WASH method is performed according to the pretreatment condition information 321 including the centrifuge process information 337 set to not perform the centrifuge process.

For example, pretreatment for measurement items such as white blood cell primary panel, AML secondary panel, B-ALL secondary panel, T-ALL secondary panel, etc. is pretreatment by the WASH method. The centrifuge process information 337 included in the pretreatment condition information 321 corresponding to the measurement items of the white blood cell primary panel and the like is set to perform the centrifugation process. On the other hand, pretreatment for measurement items such as T cell subset, lymphocyte subset and so on is pretreatment by the No-WASH method. The centrifuge process information 337 included in the pretreatment condition information 321 corresponding to the measurement items of the T cell subset and the like is set to not perform the centrifugation process.

In the embodiment, when centrifugation is set in a certain pretreatment condition information 321, a supernatant removal process and a buffer solution dispensing process are set to be performed as well. In the embodiment, when centrifugation is not set in a certain pretreatment condition information 321, a supernatant removal process and a buffer solution dispensing process also are not set.

### 2.3 Pretreatment by Pretreatment Apparatus: Example 1

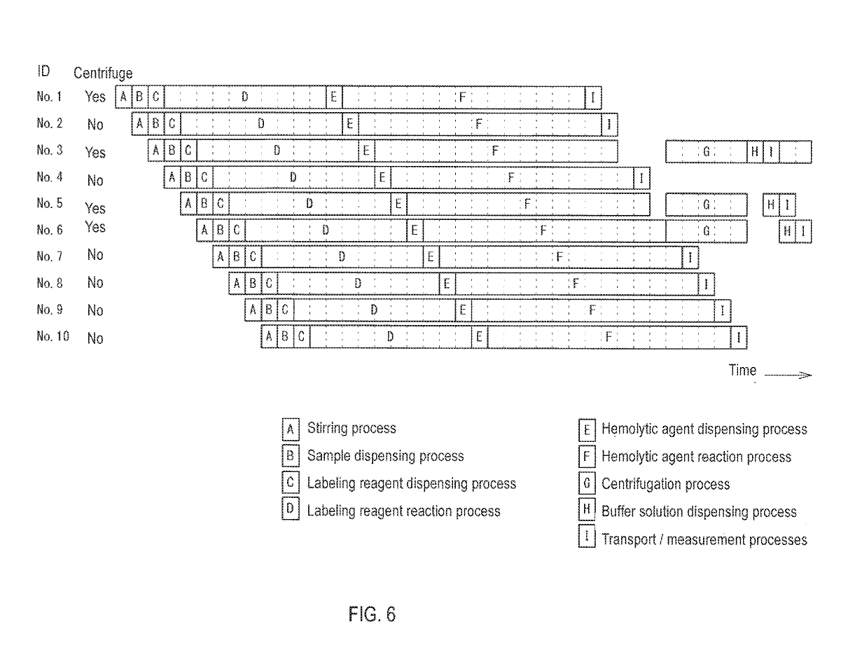

In the present embodiment, as shown in FIG. 6, the pretreatments for a plurality of samples proceed in parallel. In FIG. 6, the number of sample containers 10 that can be held in the rack 30 corresponds to 10, indicating pretreatment processes of ten samples. Among the 10 samples in FIG. 6, the sample ID indicates the second pretreatment including no centrifugation is to be performed for sample Nos. 1, 2, 4, 7-10, and the first pretreatment including centrifugation is to be performed for sample Nos. 3, 5, and 6.

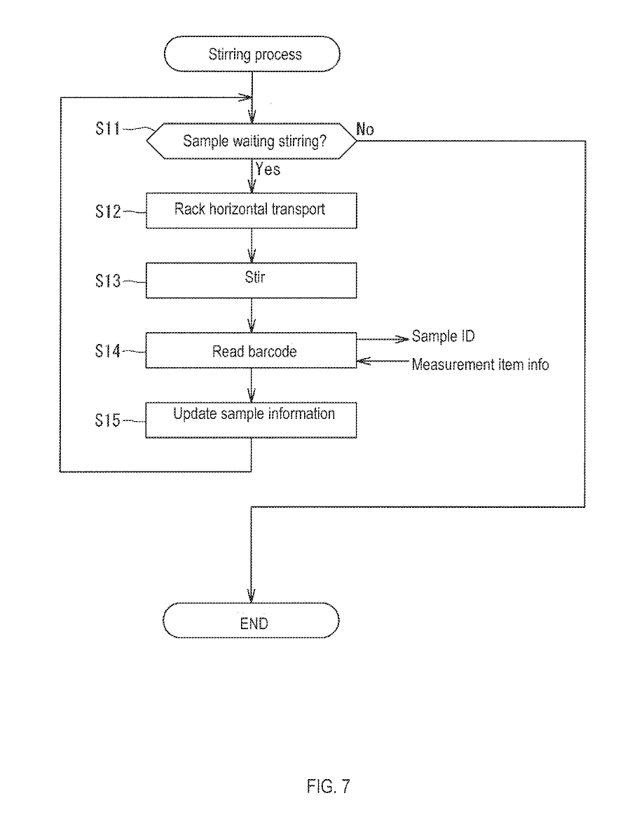

FIG. 7 to FIG. 13 show the control process sequence of each process of the pretreatments shown in FIG. 6. The processes shown in FIG. 7 to FIG. 13 are executed by the control part 300.

FIG. 7 shows the control sequence for carrying out the stirring process (step S1) shown in FIG. 4. Prior to the start of the processing in FIG. 7, it is assumed that the measurement order 701 of each of the ten samples is registered in the management computer 700 shown in FIG. 5. The measurement order 701 includes measurement item information associated with the sample ID as well as the sample ID. It is assumed that the number of sample containers 10 set in the transport part 250 also is set in the control part 300 prior to the start of the process in FIG. 7. Here, it is assumed that the fact that 10 sample containers 10 are set also is registered in the control part 300.

When the rack 30 holding the ten sample containers 10 is installed in the transport part 250 and the user specifies the pretreatment start (measurement start), the control part 300 starts the processing shown in FIG. 7 to FIG. 15.

In step S11 of FIG. 7 showing the stirring process, the control part 300 confirms the presence or absence of the sample waiting to be stirred. If the stirring and suctioning from all of the sample containers 10 set in the transport part 250 has not been completed, it is determined that there is a sample waiting to be stirred. If the stirring from all the sample containers 10 has been completed, it is determined that there is no sample waiting to be stirred, and the process in FIG. 7 ends.

If there is a sample waiting for agitation, in step S12 the control part 300 operates the transport part 250 to transversely feed the rack 30, and positions the sample container 10 containing the sample waiting for agitation to the stirring standby position 255. In step S13, the control part 300 operates the stirring part 260 and causes the stirring of the sample. The stirring is carried out by the gripping part 261 gripping the sample container 10, lifting it from the rack 30, and performing a rotating operation. When the stirring is completed, the grip art 261 returns the sample container 10 to the rack 30. When the stirred sample container 10 is returned to the rack 30, the control part 300 operates the transport part 250 to position the stirred sample container 10 at the barcode reading position 257. The barcode reader 270 reads the barcode 20 adhered to the sample container 10.

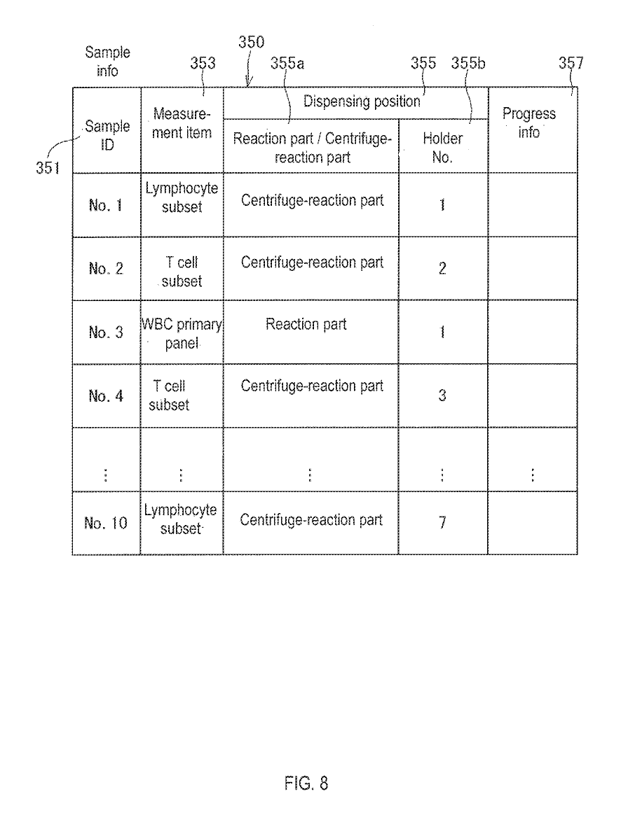

In step S14, the control part 300 obtains the sample ID by reading the barcode 20. The control part 300 accesses an external computer 700, and obtains the measurement item information corresponding to the obtained sample ID. In step S15, the control part 300 registers the acquired sample ID and measurement item information in the storage part 320 as a part of the sample information 350 shown in FIG. 8.

The sample information 350 is used by the control part 300 to manage individual samples. In the sample information 350, the sample ID 351 and the measurement item information 353 are associated with each other. The measurement item information 353 associated with the sample ID 351 is used to select the pretreatment condition information 321 to be used for pretreatment of the sample specified by the sample ID 351 from the plurality of pretreatment condition information 321. That is, the pretreatment condition information 321 having the measurement item information 331 corresponding to the measurement item information 353 of the sample information 350 is applied to the pretreatment of the sample indicated by the sample ID 351. For example, in the sample information 350, for a sample whose sample ID 351 is No. 1, a lymphocyte subset is set as measurement item information. Therefore, the pretreatment condition information 321 set for the lymphocyte subset out of the plurality of pretreatment condition information 321 is applied to the pretreatment of the sample whose sample ID is No. 1.

The information constituting the sample information 350 includes the dispensing position information 355 and the progress information 357 in addition to the sample ID 351 and the measurement item information 353. Dispensing position information 355 indicates the position at which the specimen has been dispensed. Progress information 357, has a flag indicating the completion-incompletion of each process included in the pretreatment for each sample. The information 355 and 357 will be described later.

In step S15, the control part 300 updates the progress information 357 of the stirred sample, so that the progress information 357 of that sample indicates that the stirring process has been completed. When the process up to step S15 in FIG. 7 is completed for a certain sample, the process returns to step S11, and stirring processing on the remaining samples is performed sequentially.

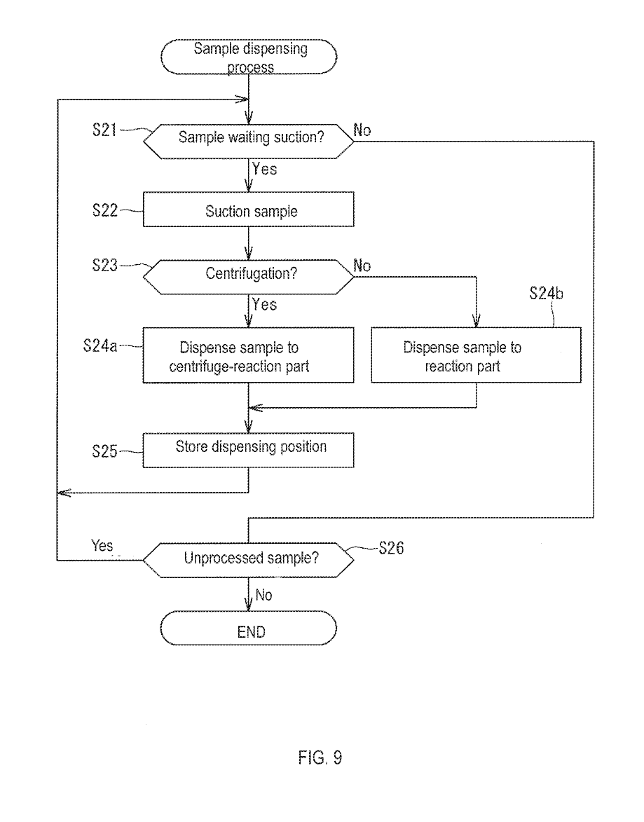

FIG. 9 shows the sample dispensing process. In step S21 of FIG. 9, the control part 300 refers to the progress information 357 to confirm whether the sample is waiting for suction. The sample waiting for suction is a sample for which the stirring process is completed but the sample dispensing process has not been completed. If it is determined that there is no sample waiting for suction, the presence or absence of a sample whose sample dispensing process has not been performed is confirmed in step S26, and there is no sample whose sample dispensing process has not been completed, that is, it is determined that the sample dispensing process of FIG. 9 is completed for all samples and the process of FIG. 9 ends. If it is determined in step S26 that there is a sample whose sample dispensing process has not been completed, the process returns to step S21.

As shown in FIG. 6, since the stirring process of each sample is sequentially completed, each sample sequentially becomes a sample waiting to be suctioned. If there is a sample waiting for suction, in step S22, the control part 300 operates the transport part 250 to position the sample container 10 containing the sample to be suctioned to the sample suction position 259.

In step S22 the control part 300 operates the sample suction part 240 to suction the whole blood sample from the sample container 10. In step S22 the sample nozzle 241 moves to the sample suction position and suctions the sample from the sample container 10.

In step 23 the control part 300 determines whether the suctioned sample is to be dispensed to the centrifuge-reaction process part 210 or dispensed to the reaction process part 220. The determination of the sample dispensing destination is performed based on whether the centrifugation process is required. For this determination, the control part 300 uses the measurement item information acquired in step S14 for the sample under determination. The control part 300 refers to the pretreatment condition information 321 containing the measurement item information 331 corresponding to the acquired measurement item information and acquires the centrifuge process information 337 included in the pretreatment condition information 321. The control part 300 determines the sample dispensing destination based on the obtained centrifuge process information 337.

If the information set in the centrifuge process information 337 indicates that the centrifuge process is to be performed, then in step S24a the control part 300 controls the sample dispensing part 240 and the centrifuge-reaction part 210 so that the suctioned sample is dispensed to the sample container 217 of the centrifuge-reaction process part 210. In step S24a, the sample nozzle 241 moves to the sample dispensing position 219a of the centrifuge-reaction process part 210, and the centrifuge-reaction process part 210 rotates to position the sample container 217 to which the suctioned sample is to be dispensed to the sample dispensing position 219a. Then the sample nozzle 241 discharges the sample into the sample container 217.

If the information set in the centrifuge process information 337 indicates that the centrifuge process is not to be performed, then in step S24b the control part 300 controls the sample dispensing part 240 and the reaction process part 220 so that the suctioned sample is dispensed to the sample container 227 of the reaction process part 220. In step S24b, the sample nozzle 241 moves to the sample dispensing position 229a of the reaction process part 220, and the reaction process part 220 rotates to position the sample container 227 to which the suctioned sample is to be dispensed to the sample dispensing position 229a. Then the sample nozzle 241 discharges the sample into the sample container 227.

In the present embodiment described above, the dispensing destination of the sample is allocated according to the necessity of centrifugation on the sample. Therefore, samples requiring centrifugation and those not requiring centrifugation may be mixed among the samples in the plurality of sample containers 10 held by the rack 30, and it is unnecessary for the user to preliminarily perform sample selection according to whether or not centrifugation is required.

In step S25 the control part 300 associates and records the dispensing position information 355 with the sample ID 351 of the dispensed sample. The dispensing position information 355 indicates which sample container 217 or 227 the sample was dispensed to among the plurality of sample containers 217 and 227. The dispensing position information 355 includes information 355a indicating whether the dispensing destination is the centrifuge-reaction process part 210 or the reaction process part 220, and information 355b indicating which of the holders 211 or 221 holds the sample containers 217 and 227. For example, the sample information 350 in FIG. 8 indicates that the dispensing position of the sample whose sample ID is No. 1 is the sample container 227 held in the holder 221 of the holder number “1” of the reaction process part 220.

When processing up to step S25 is completed for a certain sample, the control part 300 updates the progress information 357 corresponding to the sample ID 351 of the sample, and the progress information 357 of the sample indicates that the sample dispensing process has been completed. When the process up to step S25 in FIG. 9 is completed for a certain sample, the process returns to step S11, and stirring processing of the remaining samples is performed sequentially.

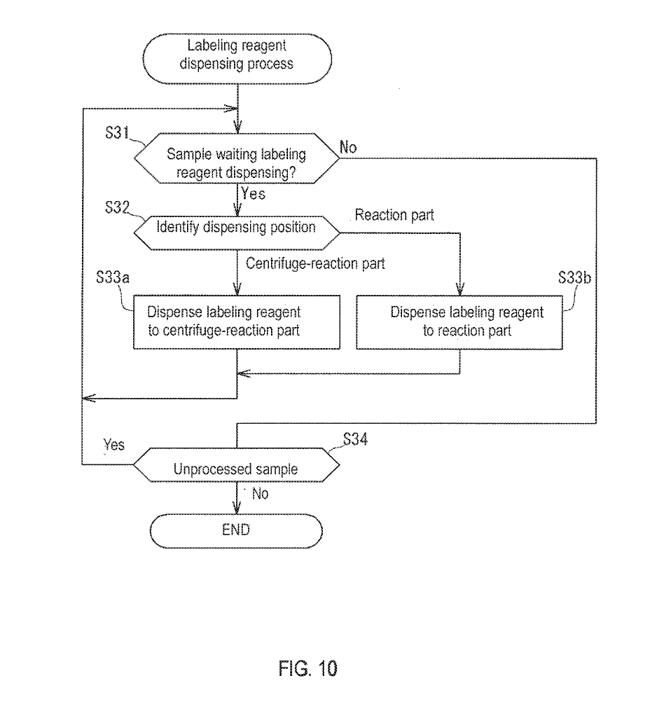

FIG. 10 shows the labeling reagent dispensing process. In step S31 of FIG. 10, the control part 300 refers to the progress information 357 to confirm whether the sample is waiting for labeling reagent dispensing. The sample waiting for labeling reagent dispensing is a sample for which the sample dispensing process is completed but the labeling reagent dispensing process has not been completed. In the labeling reagent dispensing process, the labeling reagent is dispensed to the sample containers 217 and 227 into which the sample waiting for the labeled reagent dispensing has been dispensed.

If it is determined in step S31 that there is no sample waiting for labeled reagent dispensing, the presence or absence of a sample that has not been subjected to the labeled reagent dispensing process is confirmed in step S34, and the labeled reagent dispensing process is not performed, that is, when it is determined that the labeling reagent dispensing process has been completed for all samples, the process of FIG. 10 ends. If it is determined in step S34 that there is a sample whose sample dispensing process has not been completed, the process returns to step S31.

As shown in FIG. 6, since the sample dispensing process of each specimen is sequentially completed, each sample sequentially becomes a sample waiting for labeled reagent dispensing. If there is a sample waiting for labeling reagent dispensing, in step S32, the control part 300 identifies the dispensing destination of the labeling reagent. The determination of the dispensing destination of the labeling reagent is performed based on the dispensing position information 355 for the sample waiting for the labeling reagent dispensing.

In steps S33a and S33b, the control part 300 operates the reagent dispensing part 280 and the reagent holding part 230 to suction the labeled reagent held in the reagent holder 231 of the reagent holding part 230. In steps S33a and S33b, the reagent nozzle 281 moves to the reagent suction position 239a, and the reagent holding unit 230 also rotates so that the reagent holder 231 holding the labeling reagent to be suctioned is located at the reagent suction position 239a. The labeling reagent to be suctioned is identified by the labeling reagent dispensing process information 334 of the pretreatment condition information 321 corresponding to the sample waiting for the labeling reagent dispensing. Note that a plurality of types of labeling reagent may be dispensed in steps S33a and S33b. For example, as shown in Table 1, when the measurement item is a T cell subset, there are four antigens to be measured: CD45, CD3, CD4, CD8. A labeling reagent for labeling these antigens is dispensed in steps S33a and S33b. In the labeling reagent dispensing process information 334, for each measurement item, for example, the name or identifier of the labeling reagent corresponding to the antigen is registered, as shown in Table 1 below.

TABLE 1

| Item | Antigen | | | | | | | |
|---|---|---|---|---|---|---|---|---|
| T cell subset | CD45 | CD3 | CD4 | CD8 | | | | |
| Lymphocyte | CD45 | CD3 | CD19 | CD56 | | | | |
| subset | | | | | | | | |
| Leukemia primary | CD45 | CD3 | MPO | Anti- | Intra cellular | CD22 | | |
| panel A | | | | lactoferrin | CD3 | | | |
| Leukemia primary | CD7 | CD33 | CDw65 | CD19 | HLA-DR | CD13 | Igm | CD10 |
| panel B | | | | | | | | |
| AML secondary | CD45 | Glycophorin | CD14 | CD15 | CD61 | CD64 | | |
| panel A | | | | | | | | |
| AML secondary | CD34 | CD117 | CDw65 | CD56 | CD2 | CD13 | | |
| panel B | | | | | | | | |
| B-ALL secondary | CD34 | CD22 | CD24 | CD5 | | | | |
| panel | | | | | | | | |
| T-ALL secondary | CD4 | CD8 | CD8 | CD1a | CD34 | CD34 | | |
| panel | | | | | | | | |

In steps S33a and S33b, the control part 300 dispenses the suctioned labeling reagent to the dispensing destination identified in step S32.

In the case where the dispensing destination indicated by the information 355a of the dispensing position information 355 is the centrifuge-reaction process part 210, the control part 300 operates the reagent dispensing part 280 and the centrifuge-reaction process part 210 in step S33a to dispense the suctioned labeling reagent to the sample container 217 indicated by the dispensing position information 355. In step S33a, the reagent nozzle 281 moves to the reagent dispensing position 219b of the centrifuge-reaction process part 210. In addition, the centrifuge-reaction process part 210 rotates so that the sample container 217 held by the holder 211 indicated by the holder number 355b is positioned at the reagent dispensing position 219b of the centrifuge-reaction process part 210.

In the case where the dispensing destination indicated by the information 355a of the dispensing position information 355 is the reaction process part 220, the control part 300 operates the reagent dispensing part 280 and the reaction process part 220 in step S33b to dispense the suctioned labeling reagent to the sample container 227 indicated by the dispensing position information 355. In step S33b, the reagent nozzle 281 moves to the reagent dispensing position 229b of the reaction process part 220. In addition, the reaction process part 220 rotates so that the sample container 227 held by the holder 221 indicated by the holder number 355b is positioned at the reagent dispensing position 229b of the reaction process part 220.

When processing up to step S33a and S33b is completed for a certain sample, the control part 300 updates the progress information 357 corresponding to the sample ID 351 of the sample, and the progress information 357 of the sample indicates that the labeling reagent dispensing process has been completed. When the process up to step S33a and 33b in FIG. 10 is completed for a certain sample, the process returns to step S31, and labeling reagent dispensing of the remaining samples is performed sequentially.

When the labeling reagent dispensing process for each specimen is completed, sequential standby is performed for the reaction between the sample and the labeling reagent. This standby is referred to as the labeling reagent reaction process. The standby time for the labeling reagent reaction process is in accordance with the reaction time of the labeling reagent set in the reaction process time information 336.

As shown in FIG. 6, the labeling reagent reaction process of each sample proceeds in parallel. That is, the labeling reagent reaction process in the centrifuge-reaction process part 210 and the labeling reagent reaction process in the reaction process part 220 can efficiently proceed in parallel. The same applies to the hemolytic agent reaction treatment described later.

When the labeling reagent reaction process is completed, the control part 300 updates the progress information 357 corresponding to the sample ID 351 of the sample, and the progress information 357 of the sample indicates that the labeling reagent reaction process has been completed.

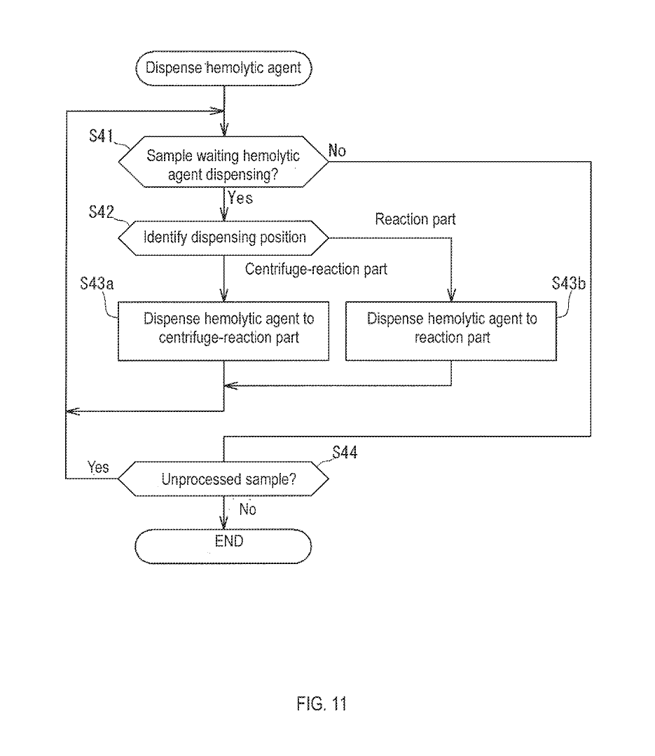

FIG. 11 shows the hemolytic agent dispensing process. In step S41 of FIG. 11, the control part 300 refers to the progress information 357 to confirm whether the sample is waiting for hemolytic agent dispensing. The sample waiting for hemolytic agent dispensing is a specimen which has completed labeling reagent reaction treatment but has not completed hemolytic agent dispensing treatment. In the hemolytic agent dispensing process, a hemolytic agent is dispensed to sample containers 217 and 227 in which samples waiting for hemolytic agent dispensing have been dispensed.

If it is determined in step S41 that there is no sample waiting for hemolytic agent dispensing, the presence or absence of a sample that has not been subjected to the hemolytic agent dispensing process is confirmed in step S44 and the hemolytic agent dispensing process is not performed, that is, when it is determined that the hemolytic agent dispensing process has been completed for all samples, the process of FIG. 11 ends. If it is determined in step S44 that there is a sample whose hemolytic agent dispensing process has not been completed, the process returns to step S41.

As shown in FIG. 6, since the labeling reagent reaction process of each sample is sequentially completed, each sample (mixture of specimen and labeling reagent) sequentially becomes a sample waiting for hemolytic agent dispensing. If there is a sample waiting for hemolytic agent dispensing, in step S42, the control part 300 identifies the dispensing destination of the hemolytic agent. The determination of the dispensing destination of the hemolytic agent is performed based on the dispensing position information 355 for the sample waiting for the hemolytic agent dispensing similarly to the dispensing destination of the labeling reagent.

In steps S43a and S43b, the control part 300 operates the reagent dispensing part 280 and the reagent holding part 230 to suction the hemolytic agent held in the reagent holder 231 of the reagent holding part 230. In steps S33a and 33b, the reagent nozzle 281 moves to the reagent suction position 239b or the reagent suction position 239c. The hemolytic agent to be suctioned is identified by the hemolytic agent dispensing processing information 335 of the pretreatment condition information 321 corresponding to the sample waiting for the hemolytic agent dispensing.

In steps S43a and S43b, the control part 300 dispenses the suctioned hemolytic agent to the dispensing destination identified in step S42. The method of dispensing the hemolytic agent is the same as the dispensing of the labeling reagent.

When processing up to step S43a and S43b is completed for a certain sample, the control part 300 updates the progress information 357 corresponding to the sample ID 351 of the sample, and the progress information 357 of the sample indicates that the hemolytic agent dispensing process has been completed. When the process up to step S43a and S43b in FIG. 11 is completed for a certain sample, the process returns to step S41, and hemolytic agent dispensing of the remaining samples is performed sequentially.

When the hemolytic agent dispensing process for each specimen is completed, the standby is sequentially performed for to the hemolysis reaction. This standby is referred to as hemolytic agent reaction process. The standby time for the hemolytic agent reaction process is in accordance with the reaction time of the hemolytic agent set in the reaction process time information 336. When the hemolytic agent reaction process between the sample and hemolytic agent is completed, the control part 300 updates the progress information 357 corresponding to the sample ID 351 of the sample, and the progress information 357 of the sample indicates that the hemolytic agent reaction process has been completed.

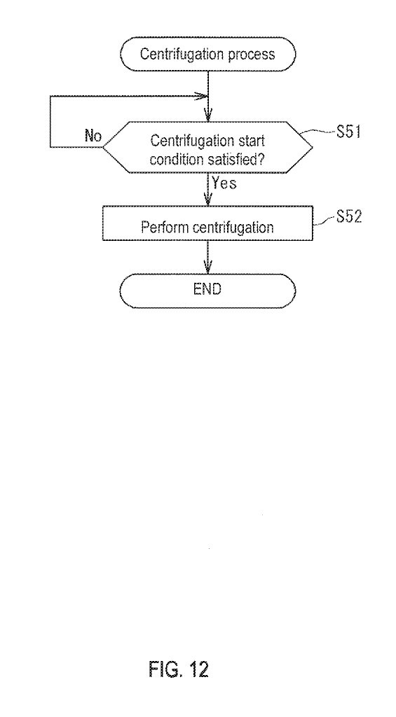

FIG. 12 shows the centrifugation process. In step S51 of FIG. 12, the control unit 300 refers to the progress information 357 and determines whether the centrifugation start condition is satisfied. The control part 300 waits until the centrifugation start condition is satisfied. When the centrifugation start condition is satisfied, the control part 300 rotates the centrifuge-reaction process part 210 and causes the centrifuge-reaction process part 210 to perform centrifugation. Centrifugation is performed on a mixed solution including a sample, a labeling reagent, and a hemolytic agent.

The centrifugation starting condition is, for example, that all samples requiring centrifugation are waiting for centrifugation. The sample waiting for centrifugation is a sample that needs to be centrifuged and the hemolytic agent reaction treatment has been completed, that is, preparation of the first mixture has been completed, but the centrifugation process has not been completed for the sample. For example, in FIG. 6, three samples with sample IDs No. 3, No. 5, and No. 6 need to be centrifuged, and when the hemolytic agent reaction treatment of the sample with the sample ID No. 6 is completed, three samples are all waiting for centrifugation. Therefore, when the hemolytic agent reaction treatment of the sample with the sample ID No. 6 is completed, the centrifugation start condition is satisfied. For samples with specimen IDs No. 3 and No. 5, even when the hemolytic agent reaction process is completed, the centrifugation process is not performed immediately and waits until the hemolytic agent reaction process of No. 6 specimen is completed.

In the present embodiment as described above, in the case where there is a plurality of samples requiring a centrifugation process among a plurality of samples requiring pretreatment, centrifugation is performed until all the plurality of samples are waiting for start of centrifugation. Centrifugation is efficiently performed since centrifugation of a plurality of samples is performed collectively after the plurality of samples are all waiting for centrifugation.

The centrifugation start condition may include a condition that a predetermined time has elapsed. For example, assume that an error occurs in one pretreatment of a sample that needs to be centrifuged and the pretreatment of that sample is canceled. In this case, all samples requiring centrifugation will not be waiting for centrifugation, but centrifugation of the samples waiting for centrifugation can be performed after a lapse of a predetermined time.

Upon completion of the centrifugation process, the control part 300 updates the progress information 357 corresponding to the sample ID 351 of one or a plurality of samples for which the centrifugation process has been completed so that progress information 357 of those samples indicates that the centrifugation process is completed.

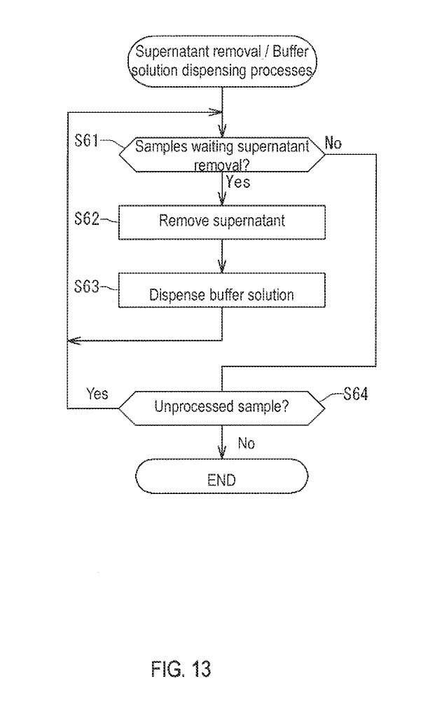

FIG. 13 shows the supernatant removal process and buffering solution dispensing process. In step S61 of FIG. 13, the control part 300 refers to the progress information 357 to confirm whether the sample is waiting for supernatant removal. The sample waiting for supernatant removal is a sample for which the centrifugation process is completed but the supernatant removal process has not been completed. In the supernatant removal treatment, the supernatant of the mixed solution after centrifugation is removed.

If it is determined in step S61 that there is no sample waiting for supernatant removal, the presence or absence of a sample whose supernatant removal treatment has not been processed is confirmed in step S64, and there is no sample whose supernatant removal treatment has not been processed yet, that is, if it is judged that the supernatant removal processing of all the samples has been completed, the process of FIG. 13 ends. If it is determined in step S64 that there is a sample whose supernatant removal process has not been completed, the process returns to step S61.

As shown in FIG. 6, since the centrifugation process for a plurality of samples is completed at the same time, after the centrifugation process all samples are waiting for the supernatant removal process. If there is a sample waiting for the supernatant removal process, in step S62 the control part 300 controls the reagent nozzle 281 to suction the supernatant of the sample container 217 and remove the supernatant. In step S61, the reagent nozzle 281 moves to the reagent dispensing position 219b. In addition, the centrifuge-reaction processing unit 210 rotates so that the sample container 217 from which the supernatant is to be removed is positioned at the reagent dispensing position 219b. The reagent nozzle 281 suctions the supernatant to remove the supernatant from the sample container 217. By using the reagent nozzle 281 for removal of the supernatant liquid instead of separately providing a suction nozzle for supernatant suction, the number of nozzles of the pretreatment device 200 can be reduced and the pretreatment device 200 can be downsized. The sample nozzle 241 also may be used for supernatant removal. The sample nozzle 241 of the embodiment is sharpened at the tip of the nozzle because of the cap piercing operation in which the lid of the sample container 10 is penetrated and suction of the sample is performed from the lateral hole provided on the side surface of the nozzle. Therefore, in order to suction the supernatant with high accuracy, a hole at the lower end of the nozzle is preferable to the lateral hole on the side of the nozzle. Since the reagent nozzle 281 of the embodiment has a suction hole at the lower end of the nozzle, it is advantageous to use the reagent nozzle 281 for removing the supernatant liquid.

In step S63, the control part 300 operates the reagent dispensing part 280 to dispense the buffer solution to the sample container 217 after removing the supernatant. In step S63, the reagent nozzle 281 suctions the buffer solution at the reagent aspiration position 239d and dispenses it to the sample container 217 located at the reagent dispensing position 219b.

Upon completion of the buffer solution dispensing process, the control part 300 updates the progress information 357 corresponding to the sample ID 351 of the sample, and the progress information 357 of the sample indicates that the supernatant removing process and the buffer solution dispensing process are completed.

### 2.4 Measurement Sample Transport and Measurement

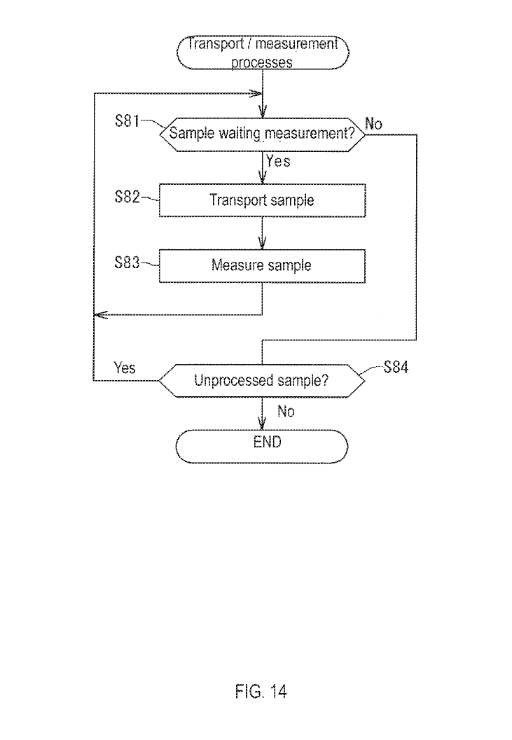

FIG. 14 shows the transfer process and measurement process of the measurement sample. In step S81 of FIG. 14, the control part 300 refers to the progress information 357 to confirm whether the sample is waiting for measurement. The sample waiting for measurement may be a sample prepared via the pretreatment by the WASH method in which pretreatment by the WASH method (up to the buffer solution dispensing process) has been completed but the transfer process and the measurement process are not completed. The sample waiting for measurement may be a sample prepared via the pretreatment by the No-WASH method in which pretreatment by the No-WASH method (up to the hemolytic agent reaction process) has been completed but the transfer process and the measurement process are not completed.

If it is determined that there is no sample waiting to be measured, the presence or absence of a sample for which the transfer process and the measurement process are completed is confirmed in step S84, and if there is no sample for which the transfer process and the measurement process are uncompleted, that is, if it is determined that the sample measurement process has been completed, the process of FIG. 14 ends. If it is determined in step S84 that there is a sample whose measurement process has not been completed, the process returns to step S81.

As shown in FIG. 6, preparation of samples is completed sequentially in accordance with the order in which preparation of samples is started for a sample which does not require centrifugation. However, for samples requiring centrifugation, preparation of the sample may be completed after a sample that started preparation later. For example, a sample (hereinafter referred to as “No. 6 sample”) prepared from a sample whose sample ID is sample No. 6 shown in FIG. 6 starts sample preparation before a sample having a sample ID of NO. 7 (hereinafter referred to as “NO. 7 sample”) which started preparation later. However, preparation of the sample of No. 7 is completed before the sample No. 6. Therefore, the sample No. 7 becomes a sample waiting for measurement before the sample No. 6.

The control part 300 performs control to transfer the samples to the measurement part in the order in which the samples wait for measurement. Therefore, the No. 7 sample is transferred to the measuring part 400 and measured by the measuring part 400 before the preparation of No. 6 sample is completed.

If there is a sample waiting for measurement, in step S82 the control part 300 operates the sample suction part 290 and the like. When the sample waiting measurement is the second sample prepared by the reaction processing part 220, the sample nozzle 291 moves to the sample suction position 229c, and the reaction process part 220 rotates so as to be position the sample container 227 containing the sample waiting to be measured at the sample suction position 229. Then, the sample nozzle 291 suctions the sample waiting for measurement from the sample container 227. The suctioned sample passes through the transfer path 600 and is transferred to the measuring part 400.

In step S83, the control part 300 controls the measuring part 400 to perform optical measurement of the transferred sample. The measuring part 400 outputs the received measurement signals to the analyzing part 500.

When the sample waiting for measurement is a sample prepared in the centrifuge-reaction processing part 210, in step S82, first the sample awaiting measurement is transferred from the sample container 217 of the centrifuge-reaction process part 210 to the sample container 227 of the reaction process part 220. For this transfer, the control part 300 operates the reagent dispensing part 280. The reagent nozzle 281 suctions the sample awaiting measurement from the sample container 217 and discharges it to the sample container 227. Thereafter, the control part 300 operates the sample suction part 290 to cause the sample nozzle 291 to suction the sample awaiting measurement in the sample container 227. The suctioned sample passes through the transfer path 600 and is transferred to the measuring part 400 where it is measured.

2.5 when the Measurement Sample is not Transported

Transfer and measurement of the measurement sample may be omitted. That is, the sample analyzer 100 may be used only for pretreatment. In cases where the transfer of the measurement sample is omitted, it is possible to cause the display device (not shown) to display indications of the sample containers 217 and 227 or the holders 211 and 221 for which the preparation of the measurement sample has been completed in the order of completion of preparation of the measurement sample. Completion of the sample preparation is when the pretreatment by the WASH method (up to the buffer solution dispensing process) is completed for the sample prepared by the WASH method, and for the sample produced by the No-WASH method is when the pretreatment by the No-WASH method (up to the hemolytic agent reaction treatment) is completed. A display indicating completion of sample preparation may be performed by lighting the vicinities of the holders 211 and 221. By displaying the sample preparation completion, the user can quickly take out the sample which has been prepared.

### 2.6 Pretreatment by Pretreatment Apparatus: Example 2

In the second example, steps S2 to S6 in FIG. 4, which are common steps in the WASH method and the No-WASH method, are performed in the reaction processing part 220. Steps S7 to S9 of FIG. 4, which is a process unique to the pretreatment of the WASH method, are performed in the centrifuge-reaction process part 210. That is, in the first pretreatment, the first half (step S2 to step S6) is performed by the reaction process part 220, and the latter half (steps S7 to S9) is performed in the centrifuge-reaction process part 210. In the second example, since the pretreatment according to the WASH method is performed in different processing parts, a step of transferring the mixed liquid being produced from the reaction process part 220 to the centrifuge-reaction process part 210 is required. As in the first example, the pretreatment of the No-WASH method is performed in the reaction process part. The points not specifically described in the second example are the same as those in the first example.

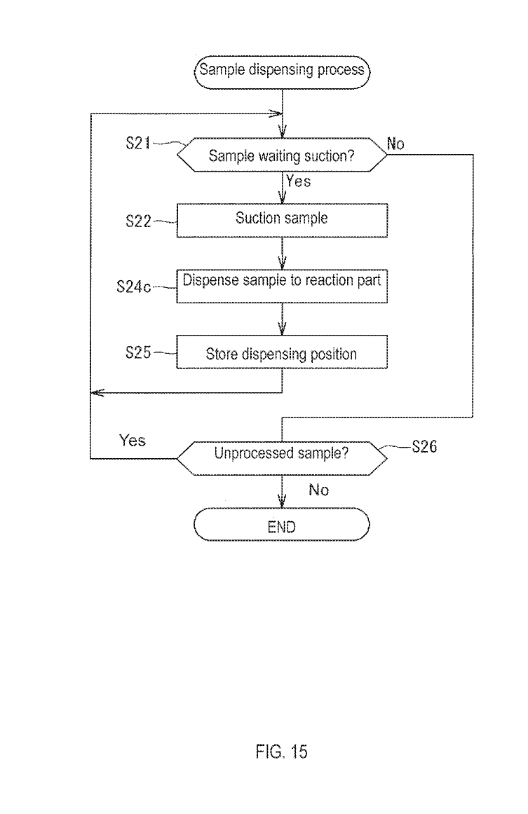

FIG. 9 shows the sample dispensing process of the second example. In step S21 in FIG. 15, when it is determined that there is a sample waiting for suction, the sample is suctioned in step S22. In step S24c, the suctioned sample is dispensed to the sample container 227 of the reaction process part 220 regardless of whether centrifugation is required.

In step S25 the control part 300 associates and records the dispensing position information 355 with the sample ID 351 of the dispensed sample. In the second example, the information 355a of the dispensing position information 355 the information indicating the dispensing destination is the reaction process part 220 for any sample.

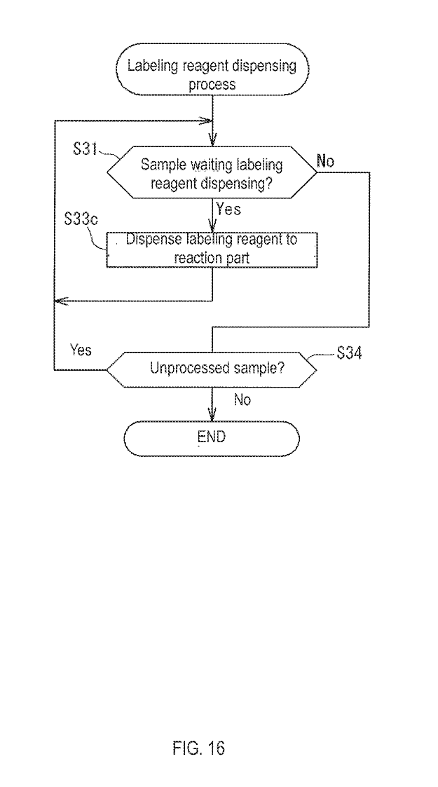

FIG. 16 shows the labeling reagent dispensing process of the second example. In step S31 of FIG. 16, when it is determined that there is a sample waiting for labeled reagent dispensing, the labeling reagent is dispensed in step S33c. In step S33, the dispensing destination of the labeling reagent is the sample container 227 of the reaction process part 220.

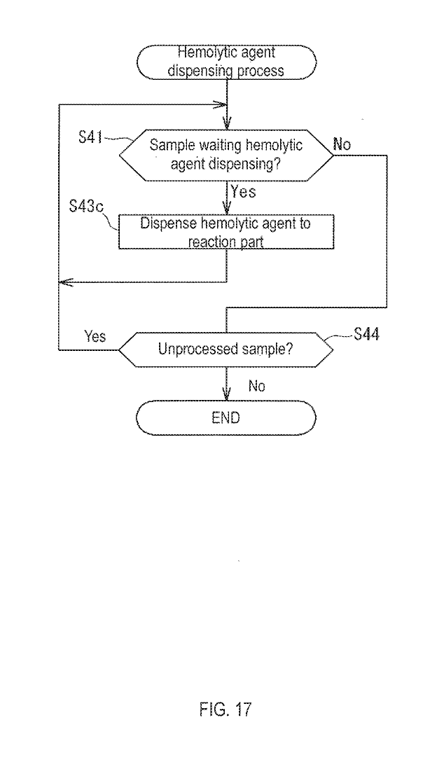

FIG. 17 shows the hemolytic agent dispensing process of the second example. [In step S41 of FIG. 17, when it is determined that there is a sample waiting for hemolytic agent dispensing, the hemolytic agent is dispensed in step S43c. In step S43, the dispensing destination of the hemolytic agent is the sample container 227 of the reaction process part 220.

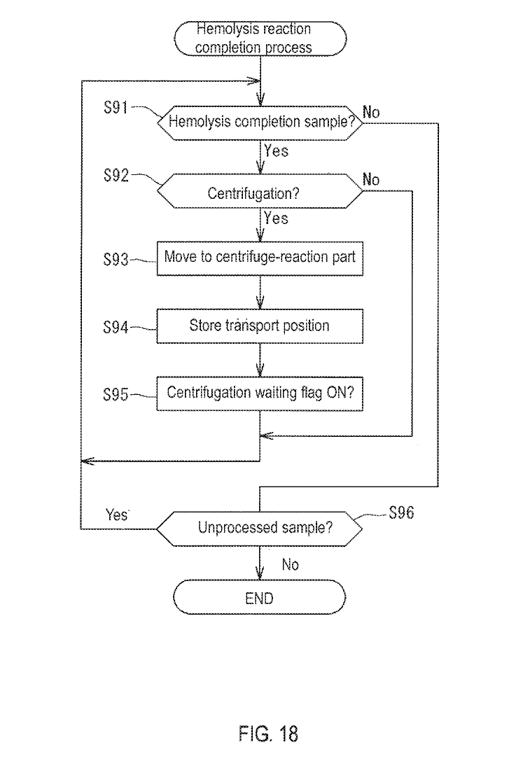

When the hemolytic agent dispensing process for each sample is completed, the hemolysis reaction process is performed sequentially. In the second example, since the treatment following the hemolytic agent reaction treatment is different from that in the first example, the hemolytic reaction completion process shown in FIG. 18 is performed. In step S91 of FIG. 18, the control part 300 refers to the progress information 357 to confirm the presence or absence of a hemolytic reaction completion sample. The hemolytic reaction completion sample is a sample for which the hemolytic agent reaction treatment has been completed.

If it is determined in step S91 that there is no hemolytic reaction completion sample, the presence or absence of a sample that has not been subjected to the hemolytic agent reaction treatment is confirmed in step S96, and thus there is no sample for which the hemolytic agent reaction treatment has not been completed, that is, when it is determined that the hemolytic agent reaction process of all the samples has been completed, the process of FIG. 18 ends. If it is determined in step S96 that there is a hemolytic agent reaction (hemolysis reaction) treatment, the process returns to step S91.

When it is determined that there is a hemolytic reaction completion sample, in step S92 the control part 300 determines whether the centrifuge treatment is required for the hemolytic reaction completion sample. In order to determine whether the centrifuge treatment is necessary, the control part 300 uses measurement item information as in step S23 of FIG. 9. The control part 300 refers to the pretreatment condition information 321 applied to the hemolytic reaction completion sample from the measurement item information and acquires the centrifuge treatment information 337 included in the pretreatment condition information 321. Based on the acquired centrifuge process information 337, the control part 300 determines whether the centrifuge process is required.

If it is determined in step S92 that centrifugation is necessary, in step S93 the control part 300 operates the reagent dispensing part 280 to move the hemolytic reaction completion sample, which is a mixture of the sample and the reagent, from the reaction process part 220 to the centrifuge-reaction process part 210.

In step S93, the reagent nozzle 281 moves to the reagent dispensing position 229b, and the reaction process part 220 rotates so that the sample container 227 containing the hemolytic reaction completion sample is positioned at the reagent dispensing position 229b. Then, the reagent nozzle 281 suctions the hemolytic reaction completion sample.

In step S93, the reagent nozzle 281 moves to the reagent dispensing position 229b, and the centrifuge-reaction process part 210 rotates so that the sample container 217 containing the hemolytic reaction completion sample is positioned at the reagent dispensing position 219b. Then, the reagent nozzle 281 discharges the suctioned hemolytic reaction completion sample to the sample container 217.

In step S94, the control part 300 stores the position at which the hemolytic reaction completion sample has been transferred as the dispensing position information 355.

In step S95, the control part 300 turns ON the centrifugation waiting flag. In the second example as well as in the first example, the centrifugation process shown in FIG. 12 is performed in the centrifuge-reaction process part 210. In the second example, the centrifugation starting condition is that all the centrifugation waiting flags for the samples requiring centrifugation are turned ON. Also in the second example, the centrifugation process for a plurality of samples is collectively performed.

In the second example as well as in the first example, a process after the centrifugation process (supernatant removal process, buffer solution dispensation process) is performed. After the pretreatment, the transfer process and measurement process may be performed.

What is claimed is:

1. A pretreatment apparatus comprising:
a sample dispensing part configured to dispense a sample containing a measurement object;
a reagent dispensing part configured to dispense labeling reagent to label the measurement object;
a first process part including: a holder configured to hold one or more first sample containers to which the sample and the labeling reagent are to be dispensed; and a centrifuge device configured to perform a centrifuge process on a mixed solution in the first sample container held by the holder;
a second process part including: a holder configured to hold one or more second sample containers to which the sample and the labeling reagent are to be dispensed; and
a control part programmed to:
control the sample dispensing part so as to dispense the sample to either the first sample container or the second sample container according to need of centrifugation process on the sample;
control the reagent dispensing part so as to dispense the labeling reagent to a sample container into which the sample is dispensed;
control the first process part to prepare a first measurement sample for flow cytometric analysis by preparing a first mixed solution in the first sample container from the sample and the labeling reagent dispensed in the first sample container, and by performing the centrifugation process on the first mixed solution; and
control the second process part to prepare a second measurement sample for flow cytometric analysis by preparing a second mixed solution in the second sample container from the sample and the labeling reagent dispensed in the second sample container.

2. The pretreatment apparatus of claim 1, wherein
the control part controls the sample dispensing part and the reagent dispensing part such that a reaction of the sample and the labeling reagent dispensed in the first sample container is progressed in parallel with a reaction of the sample and the labeling reagent dispensed in the second sample container.

3. The pretreatment apparatus of claim 1, wherein
the control part obtains information representing measurement items for the sample, and determines whether the centrifugation process is required for the sample based on the information representing measurement items.

4. The pretreatment apparatus of claim 1 further comprising:
a transport part configured to transport a sample rack holding a plurality of sample containers that contain samples;
wherein the sample dispensing part is configured to suction a sample from a sample container held in the sample rack transported by the transport part, and dispense the suctioned sample to either the first sample container or the second sample container according to the need of the centrifugation process on the sample.

5. The pretreatment apparatus of claim 1, wherein
when there are a plurality of samples requiring centrifugation process, the control part waits to start the centrifugation process until preparation of a plurality of the first mixed solutions are completed in a plurality of first sample containers, and when the preparation of the first mixed solutions are completed, controls the first process part so that centrifugation of the plurality of first mixed solutions is performed collectively.

6. The pretreatment apparatus of claim 1, wherein
the measurement object is a cell surface antigen.

7. The pretreatment apparatus of claim 1, wherein
the sample is a blood sample;
the reagent dispensing part dispenses hemolytic agent to the sample container to which the sample has been dispensed; and
the controller controls the first process part to perform the centrifugation process on the first mixed solution with which hemolytic agent has been mixed.

8. The pretreatment apparatus of claim 1, wherein
the first process part comprises a first turntable that holds a plurality of sample containers in circumferential array;
the second process part comprises a second turntable that holds a plurality of sample containers in circumferential array;
the reagent dispensing part comprises
a reagent nozzle for dispensing the labeling reagent; and
a reagent nozzle moving device configured to move the reagent nozzle along an arcing trajectory passing above the first turntable and above the second turntable.

9. The pretreatment apparatus of claim 8 further comprising:
a third turntable that holds a plurality of reagent containers;
wherein the arcing trajectory is a trajectory which passes above the third turntable.

10. The pretreatment apparatus of claim 1, wherein
the first measurement sample is a liquid which is prepared by removing a supernatant liquid from the first mixed solution after the centrifugation process.

11. The pretreatment apparatus of claim 10, wherein
the removal of the supernatant liquid is performed by at least one or the other of a sample nozzle of the sample dispensing part and a reagent nozzle of the reagent dispensing part.

12. The pretreatment apparatus of claim 10, wherein
the reagent dispensing part comprises a reagent nozzle for dispensing labeling reagent, and
the reagent nozzle is used for supernatant removal.

13. The pretreatment apparatus of claim 1, wherein
the second process part does not have a centrifuge device.

14. A sample analyzer comprising:
the pretreatment apparatus of claim 1;
a measuring part configured to optically measure the first measurement sample and the second measurement sample; and
a transport part configured to transport the first measurement sample and the second measurement sample to the measuring part.

15. The sample analyzer of claim 14, wherein
the transport part transports the second measurement sample prepared by the second process part from the second process part to the measuring part, and transports the first measurement sample prepared by the first process part to the second process part and transports the first measurement sample from the second process part to the measuring part.

16. The sample analyzer of claim 14, wherein
when the preparation of the second measurement sample that was started after the preparation of the first measurement sample is completed before the preparation of the first measurement sample is completed, the transport part transports the prepared second measurement sample to the measuring part before the preparation of the first measurement sample is completed.

17. The sample analyzer of claim 14, wherein
the measuring part is disposed above or below the first process part and the second processing part.

18. The sample analyzer of claim 14, comprising:
a first room for installing the pretreatment apparatus; and
a second room for installing the measuring part, the second room being arranged above or below the first room;
the transport part includes a sample nozzle for suctioning the first measurement sample and the second measurement sample, and a pump for transferring the measurement sample suctioned by the sample nozzle to the measuring part; and
the sample nozzle and the pump are arranged within the first room.

19. A pretreatment apparatus comprising:
a sample dispensing part configured to dispense a sample containing a measurement object;
a reagent dispensing part configured to dispense labeling reagent to label the measurement object;
a first process part including: a holder configured to hold one or more first sample containers; and a centrifuge device configured to perform a centrifugation process on a mixed solution in the first sample container held by the holder;
a second process part including a holder for holding one or more second sample containers; and
a control part programmed to:
control the sample dispensing part so as to dispense the sample to the second sample container;
control the reagent dispensing part so as to dispense the labeling reagent to the second sample container;
control the second processing unit so as to prepare a mixed solution in the second sample container from the sample and the labeling reagent dispensed in the second sample container; and
perform controls to transfer the mixed solution from the second sample container to the first sample container when a centrifugation process on the mixed solution in the second sample container is required, and perform centrifugation process on the mixed liquid by the first processing part.

20. The pretreatment apparatus of claim 19, wherein
the control part obtains information representing measurement items for the sample, and determines whether the centrifugation process is required for the sample based on the information representing measurement items.
